(19) United States
(12) Patent Application Publication
(10) Pub. No.: US 2024/0285478 A1
(43) Pub. Date: Aug. 29, 2024

(54) ESTHETIC, ROBUST, HYBRID AND BIOMIMETIC DENTAL COMPOSITION

(71) Applicant: DENTSPLY SIRONA Inc., York, PA (US)

(72) Inventor: Markus VOLLMANN, Rothenburg (DE)

(73) Assignee: DENTSPLY SIRONA Inc., York, PA (US)

(21) Appl. No.: 18/440,102

(22) Filed: Feb. 13, 2024

(30) Foreign Application Priority Data
Feb. 28, 2023 (EP) EP23159124.9

(51) Int. Cl. A61K 6/77; A61K 6/16; A61K 6/60

(52) U.S. Cl. CPC A61K 6/77; A61K 6/16; A61K 6/60

(57) ABSTRACT

The present invention relates to an esthetic, robust, hybrid, biomimetic and fast grindable and printable dental composition containing an inorganic material in the form of platelets, and an organic material. The invention also relates to dental restorations made from the dental compositions.

### BACKGROUND OF THE INVENTION

The present invention relates to an esthetic, robust, hybrid, biomimetic and fast grindable and printable dental composition.
Restoration in the sense of the present invention is a dental restoration.
Dental restoration in the sense of the present invention is a crown (including inlay, onlay and partial crown), a bridge, an implant or another kind of a dental restoration.
Restoration material in the sense of the present invention is a dental restoration material.
Platelets in the sense of the present invention are particles made of an inorganic material, having preferably a thickness of 0.2 μm-10 μm.

The prior art dental compositions often need to be grinded, milled and polished for a long time or need a temperature treatment. Zirconia needs to be sintered at the temperature of 1500° C.-1600° C. Glass ceramics needs to be crystallized at the temperature of around 800° C. In addition to this, the prior art dental compositions often suffer from short life time in vivo and/or are esthetically challenged.

The Enamic material of the VITA company suffers from esthetic disadvantages, such as high opacity and staining after a short period in vivo. It is mainly due to the microstructure, which is an interpenetrating network of inorganic skeleton framework and an infiltrated organic polymer. Other prior art CAD/CAM blocks have ‘free floating’ inorganic parts which have a high complexity of composition to adapt the refractive index to the organic material; the inorganic part composition includes a mixture of pure silica particles with the special glasses, for radio opacity, for example.

It is an object of the present invention to overcome the above shortcomings.

### DESCRIPTION OF THE INVENTION

PMMA in the sense of the present invention is Poly(methyl methacrylate).

Poly-UDMA in the sense of the present invention is Poly(urethane-dimethacrylate).

It is an object of the present invention to provide an esthetic, robust, hybrid and biomimetic dental composition, wherein the dental composition has long life time in vivo, it is fast grindable/millable without thermal post treatment. A dental restoration produced from the dental composition of the present invention requires brief polishing or applying organic based glaze only. Work-flow time for grinding, milling and polishing is less than 8 minutes, e.g. 4-5 minutes of grinding/milling and 2-3 minutes of manual and/or automatized polishing.

It is an object of the present invention to provide an esthetic, robust and hybrid dental composition for production of a dental restoration, wherein the structure and composition of the dental composition is similar to the natural tooth structure and composition, e.g. the dental composition is biomimetic. The natural tooth comprises hydroxyl-apatite crystal needles (an inorganic material) and collagen fiber (an organic material).

It is an object of the present invention to provide a dental composition comprising an inorganic material and an organic material, wherein the inorganic material is in the form of platelets.

In a preferred embodiment of the present invention, the inorganic material is a single composition high strength ceramic material present in the form of crystallized glass ceramic.

In a preferred embodiment of the present invention, the dental composition comprises 75-95 weight % of the crystallized glass ceramic.

In a preferred embodiment of the present invention, the organic material fills the voids between the platelets.

In another embodiment of the present invention, the dental composition comprises another part of the single composition high strength ceramic material in the form of ceramic grains.

In a preferred embodiment of the present invention, the ceramic grains fill the voids between the platelets.

In a preferred embodiment of the present invention, the platelets are of the thickness of 0.2 μm to 10 μm, more preferably of 0.3 μm to 7 μm, more preferably of 0.4 μm to 5 μm and even more preferably of 0.5 μm to 3 μm. The platelets have preferably the aspect ratio of from 1:1 to 1:20.

In a preferred embodiment of the present invention, the organic material is a macromolecular material. The macromolecular material is preferably selected from the group comprising poly(methacrylates), including PMMA and Poly-UDMA. In another preferred embodiment of the present invention, the organic material is selected from the group comprising Ormocer® brand materials.

The refractive index of the organic material is fitting with the refractive index of the inorganic material, which is very important for an optimal translucency of the dental composition.

In a preferred embodiment of the present invention, the difference between the refractive index of the inorganic material and of the organic material is not more than 0.1.

The distance between the platelets is in the range of several μm.

In a preferred embodiment of the present invention, the inorganic material has the composition as defined in the Table No. 1.

TABLE 1

| | From | To |
|---|---|---|
| Oxide | (weight %) | (weight %) |
| SiO₂ | 58.3 | 60.20 |
| P₂O₅ | 5.4 | 5.60 |
| Al₂O₃ | 2.5 | 2.50 |
| Li₂O | 14.4 | 14.70 |
| K₂O | 1.2 | 1.20 |
| ZrO₂ | 9.8 | 11.00 |
| CeO₂ | 0.5 | 1.90 |
| Tb₄O₇ | 1 | 1.70 |
| V₂O₅ | 0 | 0.8 |
| Er₂O₃ | 0.1 | 0.9 |
| Y₂O₃ | 0.4 | 0.4 |
| MnO₂ | 0 | 0.1 |
| Nd₂O₃ | 0 | 0.3 |
| Fe₂O₃ | 0 | 0.2 |
| B₂O₃ | 0 | 2.7 |
| Na₂O | 0.2 | 0.2 |

The dental composition of the present invention is for use for producing a dental restoration. The platelets will be crystallized to an amount of more than 60 wt % of lithium disilicate (LS2) crystals as a major crystal component combined with lithium aluminium silicates (LAS) as a minor crystal part (5-25% of all crystals).

The crystallized glass ceramic has biaxial strength (according to ISO 6872) of more than 600 MPa due to the zirconia amount diluted in the amorphous phase and the mixture of the lithium disilicate ceramic with the LAS crystals. The platelet shape of the high strength glass ceramic results in a high possible filling grade starting which is still 3D printable by an extrusion process with 75-95% vol % of crystallized glass ceramic, resulting in a significant higher strength of the hybrid dental material (wherein crystallized glass ceramic prevails in the dental composition and the organic material content is relatively minor), which exceeds the biaxial strength by at least 50% of composites available in the market as CAD machinable blocks, which have biaxial strength of around 120-270 MPa. The dental hybrid material comes closer to the composition of a real tooth.

The hybrid dental material is preferable used for CAD/CAM machinable block material and is 3D printable (due to the platelet shape of the crystallized glass-ceramic). Therefore, every internal free-form for the CAD block can be used. Due to the composition of the crystallized glass ceramic (see table above) one is able to use only this (one) kind of the crystallized glass ceramic. Other composite materials are consisting of several different glass types, merely silica (SiO2) combined with special glasses for better radio opacity.

Due to content of zirconia the radio opacity is included in the high strength glass ceramic and therefore, only one type of glass is needed.

Due to the adaptation of a relatively smaller part of the organic material in the optical refractive index within all wave-lengths of the visible light (400-800 nm), the hybrid dental material can be adjusted in translucency from transparent to nearly opaque, matching the translucency gradient of natural tooth.

The hybrid dental material of the present invention as a block material has an excellent machinability due to the small shape of the crystallized glass ceramic platelets despite the high volume percentage of this crystallized glass ceramic.

The marginal edges of the dental restoration are very sharp in comparison to pure brittle dental ceramics as pure lithium silicate CAD blocks or zirconia. Due to the high strength directly after the machining no additional thermal treatment is needed which results in a faster chair-side workflow time for the patient. By omitting the firing cycle the crown or another kind of dental restoration can be designed, milled and pace within 20 minutes when the tooth has been prepared by the dentist and an optical scan of the prepared tooth has been obtained (within 1-2 minutes).

By producing the CAD block out of this hybrid dental material, one can use an extrusion printer even with a nozzle diameter down to 50 μm due to the platelet shape of the high strength ceramic material.

The shear force during the transition of the hybrid dental material through the nozzle results in a significant lowering of the viscosity while after transition it immediately relaxes to the higher viscosity (thixotropy). The fine diameter of the nozzle can be used to mimic the natural gradient of shade translucency of the natural tooth.

The dental restoration is a crown (including an inlay, an onlay and a partial crown), a bridge, an implant or another kind of a dental restoration.

In another embodiment of the present invention, the dental restoration comprises additionally an outer organic based glaze. In a preferred embodiment of the present invention, the outer organic based glaze comprises PMMA or Poly-UDMA. The outer organic based glaze reinforces the surface roughness of the dental restoration in order to reduce abrasion of the surface by contacts with an antagonist tooth or food.

The translucency of the dental composition can be adapted from window pane translucency to semi-opacity, to adapt different translucency zones in the final dental restoration, which can therefore have a translucency gradient. When it comes to 3D-printing, different zones can be printed directly in a free shape.

1. A dental composition comprising an inorganic material and an organic material, wherein the inorganic material is in the form of platelets.

2. The dental composition according to claim 1, wherein the inorganic material is a single composition high strength ceramic material in the form of crystallized glass ceramic.

3. The dental composition according to claim 2, wherein the dental composition comprises 75-95 weight % of the crystallized glass ceramic.

4. The dental composition according to claim 1, wherein the organic material fills voids between the platelets.

5. The dental composition according to claim 2, wherein the dental composition further comprises another part of the single composition high strength ceramic material in the form of ceramic grains.

6. The dental composition according to claim 5, wherein the ceramic grains fill voids between the platelets.

7. The dental composition according to claim 1, wherein the organic material comprises a poly(methacrylate).

8. The dental composition according to claim 1, wherein the platelets have a thickness of 0.5 μm-3 μm and an aspect ratio of from 1:1 to 1:20.

9. The dental composition according to claim 1, wherein the distance between the platelets is of several μm.

10. The dental composition according to claim 1, wherein the difference between a refractive index of the inorganic material and a refractive index of the organic material is not more than 0.1.

11. The dental composition according to claim 1, wherein the translucency of the dental composition is from window pane translucency to semi-opacity.

12. A dental restoration produced from the dental composition according to claim 1.

13. The dental restoration of claim 12, wherein the dental restoration comprises a crown, a bridge, or an implant.

14. The dental restoration of claim 12, which further comprises an outer organic based glaze.
